SEQUENCE LISTING (16 sequences)

SEQ ID NO: 1 - protein, 9 aa, Homo sapiens
TLQDIVYKL

SEQ ID NO: 2 - protein, 9 aa, Homo sapiens
VRYLETSKY

SEQ ID NO: 3 - protein, 9 aa, Homo sapiens
KRYLRCPAA

SEQ ID NO: 4 - protein, 9 aa, Homo sapiens
GVALQTMKQ

SEQ ID NO: 5 - protein, 9 aa, Homo sapiens
RPPIFIRRL

SEQ ID NO: 6 - protein, 10 aa, Homo sapiens
YMCCSFLFNL

SEQ ID NO: 7 - protein, 9 aa, Homo sapiens
SQADALKYV

SEQ ID NO: 8 - protein, 9 aa, Homo sapiens
FRKAQIQGL

SEQ ID NO: 9 - protein, 9 aa, Homo sapiens
CLPSPSTPV

SEQ ID NO: 10 - protein, 9 aa, Homo sapiens
YEEEPLKDY

SEQ ID NO: 11 - protein, 9 aa, Homo sapiens
KEEVNDKRY

SEQ ID NO: 12 - protein, 9 aa, Homo sapiens
YMCSFLFNL

SEQ ID NO: 13 - protein, 9 aa, Homo sapiens
KRFRRADEV

SEQ ID NO: 14 - protein, 9 aa, Homo sapiens
YRYSQADAL

SEQ ID NO: 15 - protein, 9 aa, Homo sapiens
EELFVDYRY

SEQ ID NO: 16 - protein, 9 aa, Homo sapiens
KESRPPRKF

(19) United States
(12) Patent Application Publication
(10) Pub. No.: US 2006/0127408 A1
(43) Pub. Date: Jun. 15, 2006

(54) Cancer immunotherapy

(76) Inventors: Lawrence Sterling Young, Birmingham (GB); David Harvey Adams, Birmingham (GB); Allstair Simpson Irvine, Staffordshire (GB)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 10/538,546

(22) PCT Filed: Dec. 10, 2003
(86) PCT No.: PCT/GB03/05403

§ 371 Date: Jun. 10, 2005

(30) Foreign Application Priority Data
Dec. 11, 2002 (GB) 0228900.7

(51) Int. Cl. A61K 39/00; C07H 21/04; C12P 21/06; C07K 14/82

(52) U.S. Cl. 424/185.1; 435/69.1; 435/320.1; 435/325; 530/350; 514/44; 536/23.5

(57) ABSTRACT

The invention relates to the use of polycomb group proteins as a tumour-associated antigens. Polycomb group proteins are highly involved in body architecture development, haematopoiesis and cell cycle control. Aberrant expression of polycomb proteins has been linked with haematological malignancies, mainly lymphomas. This invention relates to the use of these polycomb group proteins as antigens for cancer immunotherapy. Immunological responses can be raised against these proteins and such responses are active against polycomb protein over-expressing tumour cells.

### FIELD OF THE INVENTION

This invention is in the field of the treatment of cancers. More specifically it is in the field of tumour-associated antigens, and the generation of anti-tumour immune responses.

### BACKGROUND OF THE INVENTION

Tumours arise due to the improper regulation of gene transcription resulting in aberrant cell growth and proliferation. This improper gene regulation results in abnormal expression of genes. Some of these genes code for tumour-associated antigens. Tumour associated antigens are proteins that are abnormally expressed in tumours, either being expressed when they are not normally, or being expressed at a level significantly higher than normal. A tumour-associated antigen is specifically a protein expressed in tumour cells that either elicits or is capable of eliciting an immune response in the tumour-bearing host.

Specific cytotoxic T cell responses against tumour associated antigens have been detected in patients and been shown to be responsible for spontaneous regressions. Many tumour-associated antigens have been identified. Tumour-associated antigens have been considered to fall into one of three main categories. The cancer/testis antigens are those that are expressed in cancers and testis. Examples of these are the MAGE family of melanoma antigens. The differentiation antigens are antigens that are normally expressed at low levels but are significantly upregulated in cancers. The differentiation antigens are associated with melanoma and examples are MART-1/MelanA and gp 100. The third type of antigens are the mutated antigens, antigens that are mutated versions of genes normally expressed. Tumour-associated antigens play an important role in the field of cancer immunotherapy.

Polycomb-group proteins play a pivotal role in development, haematopoiesis and cell cycle regulation. They form large multimeric complexes which are thought to bind to approximately 100 chromosomal sites (in Drosophila) and are involved in transcriptional repression. There have been 2 polycomb protein complexes described to date. One contains Enx/EZH2, EED, HDAC1/2 histone deacetylases and YY1, the other contains BMI-1, RING-1, HPH1, HPH2, HPC1, HPC2, HPC3 and CtBP (see Raaphorst et al. (2001) 166:5925-5934 for references). These complexes have been suggested to have opposing activities and they influence the proliferative state of cells (Raaphorst et al. (2001) 166:5925-5934). It has also been proposed that these two complexes, although both are involved in transcriptional repression, play different roles with one initiating the repression of target genes while the other maintains the repression (van Lohuizen, Curr. Opin. Genet. Dev. (1999) 9:355-361).

BMI-1 was originally discovered through studies of retroviral insertional mutagenesis in Eμ-myc transgenic mice. These mice were noted to have a significantly reduced latency period for the development of pre-B-cell lymphomas. Analysis of the sites of retroviral insertion revealed that insertion near BMI-1 was common and resulted in BMI-1 overexpression (Haupt, Y. et al. Cell (1991) 65:753-763). Consistent with this, crossing of Eμ-BMI-1 mice with Eμ-myc mice results in the accelerated onset of both T and B cell lymphomas (Haupt Y. et al. Oncogene (1993) 8:3161-3164). Thus BMI-1 is an oncogene initially described as being associated with lymphomas. BMI-1 is a polycomb group protein. BMI-1 expression tends to decrease during cell differentiation, thus highest levels are present in purified CD34+ progenitor/stem cells and there is essentially no expression in mature B and T cells (Lessard et al. Blood (1998) 91:1216-1224). Human studies have suggested that BMI-1 has a role in both B and T cell differentiation (Raaphorst et al. Am. J. Pathol. (2000) 1577:709-715; Raaphorst et al. J. Immunol. (2001) 166:5925-5934). Thus, it follows that aberrant expression of BMI-1 could result in tumour formation.

BMI-1 associates with chromatin in a cell-cycle dependent manner (Voncken et al. J. Cell Sc. (1999) 112:4627-4639).

BMI-1 misregulation has been associated with a number of cancer types including lymphomas (Beà et al. (2001)Cancer Res. 61:2409-2412; van Kemenade et al. (2001) Blood 97:3896-3901), non-small cell lung cancer (Vonlanthen et al. Br. J. Cancer (2001) 84:1372-1376) and it has been postulated that it might be involved in breast cancer due to its over expression in a number of breast cancer cell lines and its transformation of mammary epithelial cells (Dimri et al. Cancer Res. (2002) 62:47364-745). However, an analysis of BMI-1 expression in a number of haematological malignancies, as well as solid tumours, concluded that BMI-1 overexpression is most common in mantle cell lymphomas and also that ‘BMI-1 gene alterations in human neoplasms are uncommon’ (Beà et al, 2001)

RING-1, another component of the same polycomb group protein complex as BMI-1, has also been shown to have oncogeneic properties. Overexpression of RING-1 in a cell line resulted in cellular transformation as assessed by attachment independent growth and tumour formation in athymic mice (Satijn and Otte Mol. Cell Biol. (1999) 19:57-68).

Disregulation of another polycomb group protein, EZH2 (enhancer of zeste homolog 2), has also been associated with malignancy. Like BMI-1, EZH2 misregulation has been associated with haematological malignancies (van Kemenade et al. (2001) Blood 97:3896-3901). EZH2 has also been associated with non-haematopoeitic malignancies. Increasing EZH2 levels in prostate cancer have been correlated both with disease stage and prognosis (Varambally et al. Nature (2002) 419:624-629).

International patent application WO 01/53834 (Otte/University of Amsterdam) describes methods of identifying and characterising tumour cells, by means of analysing their expression of proteins of the polycomb group complex and, in particular, BMI-1.

Thus the involvement of polycomb group proteins in tumorigenesis, especially in the haematopoietic system is well described in the prior art, however the use of these proteins as tumour-associated antigens has not been described. Due to the level of expression of these proteins in normal tissues those skilled in the art would presume that they are not good candidate tumour associated antigens. Also disclosed in the current application is the observation that BMI-1 disregulation, particularly upregulation, is far more common than previously reported, and is detectable in a large range of non-haematological malignancies.

### SUMMARY OF THE INVENTION

The present invention relates to the use of polycomb proteins as antigens for the immunotherapeutic treatment of cancer. Polycomb group proteins, especially BMI-1 and EZH2, have been shown to be aberrantly expressed in a number of cancers. However these proteins are also expressed in many normal tissues.

We demonstrate in the examples below that both humoral and cellular immune responses to polycomb proteins can be detected in the sera and peripheral blood mononuclear cell preparations from both cancer patients and healthy donors. Thus self-immune responses to these antigens can be generated in humans and these responses, at least at the levels detected appear not to have any pathological consequences. We also demonstrate that in a murine model significant anti-polycomb protein responses can be induced by vaccination. Antibody and T cell responses can be detected and these responses can have anti-tumour cell activity.

We have also shown that the expression of at least one polycomb protein, BMI-1, is highly upregulated in a large number of human tumour types.

Immunisation against tumour-associated antigens can be achieved using a number of approaches. These approaches can include but are not limited to genetic immunisation, protein vaccination, peptide vaccination, cell-based vaccination (especially dendritic cell vaccination), vaccination with virus-like particles (VLPs).

Genetic vaccination has been used in numerous preclinical cancer immunotherapy studies and in cancer immunotherapy trials. Genetic vaccination involves vaccination by delivering the genes encoding the antigen to which an immune response is required into cells of an individual(Ulmer et al. Curr. Opin. Immunol. (1996) 8:531-536). The genes can be delivered as plasmid DNA, either ‘naked’ or formulated to improve DNA transfection with lipids, etc., by simple intramuscular injection (Ulmer et al. Science 1993 259:1745-1748), by intradermal injection (Raz et al. Proc. Natl. Acad. Sci. 1994 91:9519-9523), by intranasal administration (Klavinskis et al J. Immunol. 1999 162:254-262; Sasaki et al. J. Virol. 1998 72:4931-4939), or by topical application (Fan et al. Nature Biotech 1999 17:870-872). Alternatively the genes can be delivered as plasmid DNA coated onto high density (usually gold) beads that are ‘fired’ into the skin of an individual using a gene gun (Fynan et al. Proc. Nat. Acad. Sci. 1993 90:11478-11482; Fuller et al. J. Med. Primatol. 1996 25:236-241), as plasmid DNA in microparticles (Chen et al. J. Virology 1998: 5757-5761), as a eukaryotic expression construct in a bacterial delivery system (Sizemore et al, 1995, Science 270: 299-302; Paglia et al, 1998, Blood 92: 3172-3176) or in a recombinant virus, the virus being directly administered into an individual. Recombinant viruses used for genetic immunisation include, but are not limited to, adenovirus (Fooks et al. Virology 1995 210:456-465; Imler J-L Vaccine 1995 13:1143-1151; Chen et al. J. Immunol 1996 156:224-231; Rosenberg et al. J. Natl. Cancer Inst. 1998 90:1894-1900), and pox viruses such as vaccinia virus (Epstein et al. J. Immunol. 1993 150:5484-5493; Graham et al. J. Infect. Dis. 1992 166:244-252).

Responses to genetic immunisation procedures can be boosted by modification of the antigen. This modification can take the form of ubiquitination to improve antigen degradation and thus presentation (Rodriguez et al. J. Virology 1997 71:8497-8503; Fu et al. Vaccine 1998 16:1711-1717) and targeting antigen to sites or cells key to immune induction (Boyle et al. Nature 1998 392:408-411; Deliyannis et al. Proc. Natl. Acad. Sci. 2000, 97: 6676-6680)

Protein vaccination usually involves the administration of recombinant protein antigen in combination with an adjuvant. Protein vaccines are usually administered by the intramuscular, subcutaneous or intradermal routes (Vaccine Design, the subunit and adjuvant approach, Eds: Powell and Newman, Plenum Press, New York, 1995, and references therein).

Peptide vaccination involves vaccination with peptide epitopes from antigens. These peptides are usually MHC or HLA Class I-binding peptides designed to elicit CD8+ve cytotoxic T cell responses, however they may also include Class II restricted peptides. Peptide vaccination has shown anti-tumour efficacy in preclinical tumour models (Schallert et al, Eur. J. Immunol. 2002 32:752-760; Takigawa et al. Ann. N.Y. Acad. Sci. 2001 941:139-146; Lo-Man et al. J. Immunol. 2001 166:2849-2854), and has been tested in anti-cancer clinical trials (Jäger et al. Int. J. Cancer 1996 66:162-169; Jäger et al. Int. J. Cancer 1996 67:54-62; Rosenberg et al. Nature Medicine 1998 4:321-326).

Since the recent development of protocols for the generation of large numbers of dendritic cells in vitro/ex vivo, immunotherapy with ex vivo manipulated dendritic cells has become a promising field for cancer treatment (reviewed in Fong & Engleman Ann. Rev. Immunol. 2000 18:245-273). Dendritic cells can be ‘loaded’ with defined tumour-associated antigen using methodologies including, but not limited to, protein pulsing (Hsu et al. Nature Medicine (1996) 2:52-58; Reichardt et al. Blood (1999) 93:2411-2419), peptide-pulsing (Tjoa et al. The Prostate (1997) 32:272-278; Morse et al. Clinical Cancer Research (1999) 5:1331-1338; Thurner et al. J. Exp. Med. (1999) 190:1669-1678; Mackensen et al. Int. J. Cancer (2000) 86:385-392), DNA transfecting (Irvine et al. Nat. Biotech. (2000) 18:1273-1278; Lohmann et al. Cancer Gene Therapy (2000) 7:605-614), virally transducing (Ishida et al. Clin. Exp. Immunol. (1999) 117:224-251; Kaplan et al. J. Immunol (1999) 163:699-707; Yang et al. J. Immunol (2000) 164:4204-4211), and RNA-pulsing (Boczkowski et al. Cancer Research (2000)60:1028-1034; Heiser et al. J. Clin. Invest. (2002) 109:409-417).

BMI-1 is a self-protein and thus in order to generate an immune response to it tolerance must be broken. Thus to boost the generation of anti-BMI-1 immune responses a number of approaches can be taken, these approaches are well known to those skilled in the art. These approaches include but are not limited to, inclusion of non-self helper epitopes in the immunogen (Dalum et al. Nat. Biotech 1999 17:666-669), fusion of the antigen to a strong foreign antigen (King et al. Nature Medicine 1998 4:1281-1286), or use of a xenogeneic version of the antigen (Naftzger et al, Proc. Natl. Acad. Sci. 1996 93:14809-14814). All of these approaches are applicable to most, if not all, of the immunisation methodologies described above.

Optimal cancer therapies may involve the combination of BMI-1 with other tumour-associated antigens, thus increasing the breadth and hopefully potency of the immune response and limiting the chance of immune escape by tumour-associated antigen downregulation.

Accordingly, the invention provides, in a first aspect, a polycomb protein, or an immunogenic peptide or epitope derived therefrom, or an isolated polynucleotide encoding said protein, peptide or epitope, for use as a medicament in the immunotherapy of cancer. Preferably, the protein is selected from the list consisting of Enx/EZH2, EED, BMI-1, RING-1, HPH1, HPH2, HPC3 and CtBP, more preferably it is Enx/EZH2 or BMI-1, most preferably it is BMI-1.

Preferably, the cancer to be treated is derived from a tissue or organ selected from the list consisting of; liver, lung, breast, stomach, cervix, prostate, bladder, pancreas, brain, colorectal or ovary, or is a melanoma, lymphoma or leukaemia.

In a second aspect, the invention provides a vector comprising a polynucleotide encoding such a protein, or immunogenic peptide or epitope derived therefrom.

The vector may be any vector capable of transferring DNA to a cell. Preferably, the vector is an integrating vector or alternatively a non-integrating vector.

Preferred integrating vectors include recombinant retroviral vectors. A recombinant retroviral vector will include DNA of at least a portion of a retroviral genome which portion is capable of infecting the target cells. The term “infection” is used to mean the process by which a virus transfers genetic material to its host or target cell. Preferably, the retrovirus used in the construction of a vector of the invention is also rendered replication-defective to remove the effect of viral replication on the target cells. In such cases, the replication-defective viral genome can be packaged by a helper virus in accordance with conventional techniques. Generally, any retrovirus meeting the above criteria of infectivity and capability of functional gene transfer can be employed in the practice of the invention. Lentiviral vectors are especially preferred.

Suitable retroviral vectors include but are not limited to pLJ, pZip, pWe and pEM, well known to those of skill in the art. Suitable packaging virus lines for replication-defective retroviruses include, for example, ΨCrip, ΨCre, Ψ2 and ΨAm.

Other vectors useful in the present invention include adenovirus, adeno-associated virus, SV40 virus, vaccinia virus, HSV and poxvirus vectors. A preferred vector is the adenovirus. Adenovirus vectors are well known to those skilled in the art and have been used to deliver genes to numerous cell types, including airway epithelium, skeletal muscle, liver, brain and skin (Hitt, M M, Addison C L and Graham, F L (1997) Advances in Pharmacology 40: 137-206; Anderson W F (1998) Nature 392: (6679 Suppl): 25-30.).

A further preferred vector is the adeno-associated (AAV) vector. AAV vectors are well known to those skilled in the art and have been used to stably transduce human T-lymphocytes, fibroblasts, nasal polyp, skeletal muscle, brain, erythroid and haematopoietic stem cells for gene therapy applications (Philip et al (1994). Mol Cell Biol 14: 2411-2418; Russell et al (1994) Proc Natl Acad Sci USA 91: 8915-8919; Flotte T R, Afione S A, Conrad C, McGrath S A, Solow R, Oka H., Zeitlin P L, Guggino W B and Carter B J (1993). Proc Natl Acad Sci USA 90: 10613-10617; Walsh et al (1994). Proc Natl Acad Sci USA 89: 7257-7261; Miller et al(1994) Proc Natl Acad Sci USA 91:10183-10187; Emerson (1996). Blood 87, 3082-3088). International Patent Application WO 91/18088 describes specific AAV based vectors.

Preferred episomal vectors include transient non-replicating episomal vectors and self-replicating episomal vectors with functions derived from viral origins of replication such as those from EBV, human papovavirus (BK) and BPV-1. Such integrating and episomal vectors are well known to those skilled in the art and are fully described in the body of literature well known to those skilled in the art. In particular, suitable episomal vectors are described in WO98/07876.

Mammalian artificial chromosomes can also be used as vectors in the present invention. The use of mammalian artificial chromosomes is discussed by Calos (1996 Trends in Genetics 12: 463-466).

In a preferred embodiment, the vector of the present invention is a plasmid. The plasmid may be is a non-replicating, non-integrating plasmid.

The term “plasmid” as used herein refers to any nucleic acid encoding an expressible gene and includes linear or circular nucleic acids and double or single stranded nucleic acids. The nucleic acid can be DNA or RNA and may comprise modified nucleotides or ribonucleotides, and may be chemically modified by such means as methylation or the inclusion of protecting groups or cap- or tail structures.

A non-replicating, non-integrating plasmid is a nucleic acid which when transfected into a host cell does not replicate and does not specifically integrate into the host cell's genome (i.e. does not integrate at high frequencies and does not integrate at specific sites). As used herein, ‘host cell’ refers to any cell used the replicate a vector, or to express a product from such a vector. It does not imply any particular source of the cell.

Replicating plasmids can be identified using standard assays including the standard replication assay of Ustav et al. (1991 EMBO J 10: 449-457).

Numerous techniques are known and are useful according to the invention for delivering the vectors described herein to cells, including the use of nucleic acid condensing agents, electroporation, complexing with asbestos, polybrene, DEAE cellulose, Dextran, liposomes, cationic liposomes, lipopolyamines, polyornithine, particle bombardment and direct microinjection (reviewed by Kucherlapati and Skoultchi, 1984 CRC Crit. Rev. Biochem 16: 349-379; Keown et al., 1990 Methods Enzymol 185: 527-37).

A vector of the invention may be delivered to a host cell non-specifically or specifically (i.e., to a designated subset of host cells) via a viral or non-viral means of delivery. Preferred delivery methods of viral origin include viral particle-producing packaging cell lines as transfection recipients for the vector of the present invention into which viral packaging signals have been engineered, such as those of adenovirus, herpes viruses and papovaviruses. Preferred non-viral based gene delivery means and methods may also be used in the invention and include direct naked nucleic acid injection, nucleic acid condensing peptides and non-peptides, cationic liposomes and encapsulation in liposomes.

The direct delivery of vector into tissue has been described and some short-term gene expression has been achieved. Direct delivery of vector into muscle (Wolff et al., 1990) thyroid (Sikes et al., 1994 Human Gene Therapy 5: 837-844.) melanoma (Vile et al., 1993 Vile R G and Hart I R (1993, Cancer Res 53: 962-967.), skin (Hengge et al., 1995, Nature Genet 10: 161-166.), liver (Hickman et al., 1994, Human Gene Therapy 5: 1477-1483.) and after exposure of airway epithelium (Meyer et al., 1995, Gene Therapy, 2, 450-460, 1995) is clearly described in the prior art.

Various peptides derived from the amino acid sequences of viral envelope proteins have been used in gene transfer when co-administered with polylysine DNA complexes (Plank et al.,1994, J Biol Chem 269: 12918-12924.; Trubetskoy et al.,1992, Bioconjugate Chem 3: 323-327; WO 91/17773; WO 92/19287) and Mack et al., (1994, Am J Med Sci 307: 138-143.) suggest that co-condensation of polylysine conjugates with cationic lipids can lead to improvement in gene transfer efficiency. International Patent Application WO 95/02698 discloses the use of viral components to attempt to increase the efficiency of cationic lipid gene transfer.

Nucleic acid condensing agents useful in the invention include spermine, spermine derivatives, histones, cationic peptides, cationic non-peptides such as polyethyleneimine (PEI) and polylysine. ‘Spermine derivatives’ refers to analogues and derivatives of spermine and include compounds as set forth in International Patent Application WO 93/18759 (published Sep. 30, 1993).

Disulphide bonds have been used to link the peptidic components of a delivery vehicle (Cotten et al., 1992, Enzymol 217: 618-644.); see also Trubetskoy et al. (supra).

Delivery vehicles for delivery of DNA constructs to cells are known in the art and include DNA/poly-cation complexes which are specific for a cell surface receptor, as described in, for example, Wu and Wu, 1988, J Biol Chem 263:14621; Wilson et al., 1992, J Biol Chem 267: 963-967; and U.S. Patent No. 5,166,320.

Delivery of a vector according to the invention is contemplated using nucleic acid condensing peptides. Nucleic acid condensing peptides, which are particularly useful for condensing the vector and delivering the vector to a cell, are described in International Patent Application WO 96/41606. Functional groups may be bound to peptides useful for delivery of a vector according to the invention, as described in WO 96/41606. These functional groups may include a ligand that targets a specific cell-type such as a monoclonal antibody, insulin, transferrin, asialoglycoprotein, or a sugar. The ligand thus may target cells in a non-specific manner or in a specific manner that is restricted with respect to cell type.

The functional groups also may comprise a lipid, such as palmitoyl, oleyl, or stearoyl; a neutral hydrophilic polymer such as polyethylene glycol (PEG), or polyvinylpyrrolidine (PVP); a fusogenic peptide such as the HA peptide of influenza virus; or a recombinase or an integrase. The functional group also may comprise an intracellular trafficking protein such as a nuclear localisation sequence (NLS), an endosome escape signal such as a membrane disruptive peptide, or a signal directing a protein directly to the cytoplasm.

In a further aspect, the invention provides a host cell transfected or transduced with, or otherwise containing, the isolated polynucleotide or vector comprising such a polynucleotide of the present invention. Preferably it is an antigen presenting cell. More preferably, it is a dendritic cell and, most preferably, it is an autologous cell derived from the patient and transfected or transduced either in vivo or ex vivo.

Alternatively, the host cell may be loaded or pulsed so that it contains the protein of the invention, or an immunogenic peptide or epitope derived therefrom. As will be understood by those of skill in the art, in such circumstances antigen presentation by means of the MHC Class II mechanism is favoured, whilst with delivery of the antigen to the cell in the form of an expressible polynucleotide, presentation via the MHC Class I mechanism is favoured. In preferred embodiment, both approaches are used, with autologous dendritic cells both loaded with peptide antigen and transfected with expressible polynucleotide encoding the same or different peptides or antigens as part of an ex vivo procedure before being returned to the patient.

In a further aspect, the invention provides a vaccine composition comprising the protein, peptide, epitope, polynucleotide, vector or host cell as described above, together with a pharmaceutically acceptable excipient, carrier, buffer or adjuvant.

In one preferred embodiment, the vaccine composition is a protein vaccine composition comprising the protein, peptide or epitope of the invention, together with a pharmaceutically acceptable excipient, carrier, buffer or adjuvant. Alternatively, it is a DNA vaccine composition comprising the polynucleotide or vector of the invention, together with a pharmaceutically acceptable excipient, carrier, buffer or adjuvant.

Also provided is the use of the protein, peptide, epitope, polynucleotide, vector or host cell of the invention as described above, for the manufacture of a medicament for the immunotherapy of cancer. Preferably, such use is for the manufacture of a medicament for the treatment of a cancer derived from a tissue or organ selected from the list consisting of; liver, lung, bladder, pancreas, brain, stomach, cervix, prostate, colorectal or ovary, or is a melanoma, lymphoma or leukaemia.

In a final aspect, the invention provides a method of treating a cancer by immunotherapy, comprising administering to a patient a vaccine composition comprising a polycomb protein, or an immunogenic peptide or epitope derived therefrom, or an isolated polynucleotide encoding said protein, peptide or epitope.

Alternatively, the method comprises administering to a patient a vaccine composition comprising a polynucleotide encoding a polycomb protein, or an immunogenic peptide or epitope derived therefrom. Preferably, the polynucleotide is included in a vector. More preferably the vector is an integrating vector. Alternatively it is a non-integrating vector. In either case, it is preferred that the vector is a viral vector, as described above.

In a preferred embodiment of the method, the protein is selected from the list consisting of Enx/EZH2, EED, BMI-1, RING-1, HPH1, HPH2, HPC3 and CtBP. More preferably, it is Enx/EZH2 or BMI-1. Most preferably, it is BMI-1.

In another preferred embodiment, the method comprises administering to a patient a vaccine composition comprising a host cell containing a polycomb protein, or an immunogenic peptide or epitope derived therefrom, or an isolated polynucleotide or vector encoding said protein, peptide or epitope. Preferably the cell is a dendritic cell, more preferably an autologous dendritic cell.

It is preferred that the cancer to be treated is derived from a tissue or organ selected from the list consisting of; liver, lung, breast, stomach, cervix, prostate, bladder, pancreas, brain, colorectal or ovary, or is a melanoma, lymphoma or leukaemia.

### DETAILED DESCRIPTION OF THE INVENTION

### DESCRIPTION OF THE FIGURES

The invention will now be described with reference to the following figures.

FIG. 1 shows an example of a serum sample from an HCC (hepatocellular carcinoma) patient reacting with different HCC-derived cDNA clones. Clones 45 and 56 represent clones carrying different portions of BMI-1.

FIG. 2 shows ELIspot results examining whether there are T cell responses to predicted BMI-1 epitopes in PBMC preparations from hepatocellular carcinoma patients. The peptides are: a predicted A0201 peptide—‘TLQ’ (TLQDIVYKL; SEQ ID NO: 1); the predicted B2702/05 peptides—‘VRY’ (VRYLETSKY; SEQ ID NO:2) and ‘KRY’ (KRYLRCPAA; SEQ ID NO:3); an HLA-A02-restricted epitope from alpha-fetoprotein—‘GVA’ (GVALQTMKQ; SEQ ID NO:4); and a B7-restricted peptide from EBNA3a (negative control)—‘RPPI’ (RPPIFIRRL; SEQ ID NO:5); ‘T only’ is T cells only and ‘PBL’ is peripheral blood lymphocytes alone.

FIG. 3 shows ELIspot results examining whether there are T cell responses to predicted BMI-1 epitopes in PBMC preparations from a normal volunteer and an hepatocellular carcinoma patient. PBMC were untreated or infected with the adenoviruses indicated (ADE3C, EBNA3c expressing adenovirus; ADBGAL, β-galactosidase expressing adenovirus, ADBMI, BMI-1 expressing adenovirus).

FIG. 4 shows ELIspot results examining whether there are T cell responses to predicted EZH2 epitopes in PBMC preparations from normal volunteers. The peptides are the predicted A0201 peptides, YMC—YMCCSFLFNL; (SEQ ID NO:6); SQA—SQADALKYV; (SEQ ID NO:7) or FRK—FRKAQIQGL (SEQ ID NO:8) an HLA-B27 restricted peptide from EBNA3c (negative control).

FIG. 5 shows immunohistochemical staining of tumour sections for BMI-1. The sections shown are A) hepatocellular carcinoma (arrow N indicates positively staining nuclei of hepatocellular carcinoma cells), B) gastric cancer (arrow S indicates stromal cells showing significantly less staining, arrow C indicates glandular epithelial gastric cancer cells staining positive), C) breast cancer (arrow B indicates breast cancer cells staining highly positive for BMI-1) and D) prostate cancer (arrow P indicates prostate cancer cells staining strongly for BMI-1 compared to surrounding poorly/negatively staining fibroblasts).

FIG. 6 shows CTL responses to EZH2 polycomb protein epitopes in liver cancer patients (A) and normal controls (B) as measured by ELISPOT assays.

FIG. 7 shows the results of chromium release assays demonstrating EZH2 peptide-specific CTL lysis of target cells.

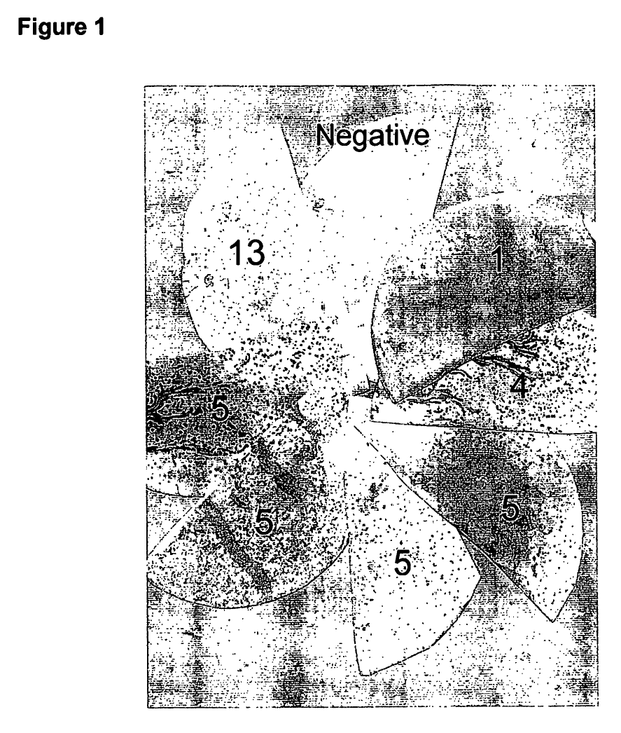

### EXAMPLE 1

### Humoral Responses to BMI-1

Method

The serological detection of antigens using a recombinant cDNA expression library derived from a hepatocellular carcinoma (HCC, purchased from Novagen and confirmed with our own ‘in house’ library) was performed using serum from HCC patients and normal healthy controls. The sera were preabsorbed against both E.coli lysate and a phage λ lysate and then used to probe filters carrying the cDNA library. Briefly, a culture of BL21 cells are grown overnight and resuspended in 10 mM MgSO₄. 600 ul of these cells are incubated with 3 μl of the cDNA HCC library for 15 minutes and then mixed with NZY agarose and plated out onto NZY agar plates. These are incubated for 6-8 hours until plaques are visible and then overlaid with IPTG soaked Hybond-C filters. The plates are stored overnight at 4° C. and incubated at 37° C. for 4 hours the next day. The filters are removed from the plates, washed 3 times in TBS-T and then blocked for an hour in TBS-T 5% milk. They are then washed again before incubating with the absorbed serum (1 in 1000 dilution in TBS-T 5% milk) for 2.5 hours room temperature, followed by washing and then incubating with the second antibody 1 in 1000 (goat anti-human IgG alkaline phosphatase conjugate, Sigma) for 2 hours at room temperature. Filters are washed again and stained using the Biorad alkaline phosphatase kit for no more than 10 minutes. Once the filters have dried positive plaques are identified and picked from the plates, eluted, rescreened and cloned to monoclonality. The insert is screened to check it is not patient specific by screening with a number of different patient sera and with normal sera. Screening controls include second step antibody alone to exclude anti-immunoglobulin responses and screening alongside a negative clone. Positive clones are then excised and sequenced. The sequences are compared to known sequences using the BLAST nucleotide search on the NCBI database.

Results

Three sequences were identified as different cDNA clones of BMI-1 using this approach. All 9 HCC patients screened had strong antibody responses to at least one of the clones. Normal donors were also screened and 5 out of 6 had responses to BMI-1 but these were weaker than those observed in the HCC patients.

### EXAMPLE 2

### Prediction of HLA-A2 Epitopes in BMI-1 and ELISpot Studies

Epitopes were predicted from the BMI-1 protein sequence using the BIMAS (BioInformatics and Molecular Analysis Section) web site (Parker, KC et al 1994 Scheme for ranking potential HLA-A2 binding peptides based on independent binding of individual peptide side-chains. J Immunol 152:163). The peptides were selected according to scores compared to known epitopes from Epstein-Barr virus.

| | | |
|---|---|---|
| A0201 peptides | TLQDIVYKL | (SEQ ID NO: 1) |
| and | | |
| | CLPSPSTPV; | (SEQ ID NO: 9) |
| B2702/05 peptides | VRYLETSKY | (SEQ ID NO: 2) |
| and | | |
| | KRYLRCPAA; | (SEQ ID NO: 3) |
| B4402/03 peptides | YEEEPLKDY | (SEQ ID NO: 10) |
| and | | |
| | KEEVNDKRY. | (SEQ ID NO: 11) |

Elispots were performed using these peptides on peripheral blood mononuclear cells (PBMCs) from HCC patients and normal donors. Frozen PBMC were recovered overnight before use and the assays were performed in duplicate where possible using 2-4×10⁵ cells per well. Peptides were used at concentration of 100 μg/ml adding 10 μl per well. The final well volume was 100 μl. The plates were incubated overnight and then washed, antibodies added and stained according to usual protocols. The plates were counted using an automated system (AID, Strassburg, Germany).

Results

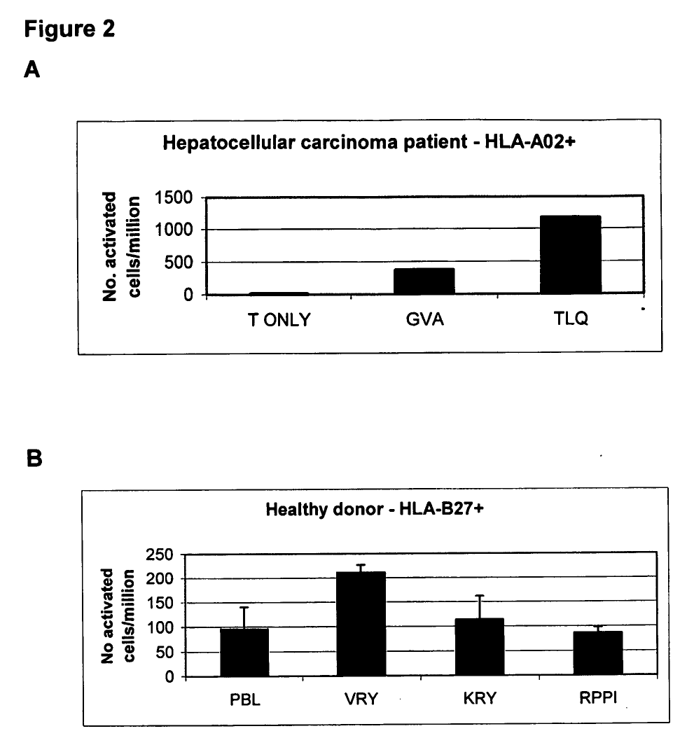

Specific T cell responses (IFNγ secretion) can be seen to a single HLA-AO2 restricted peptide in an HLA-AO2 patient (FIG. 2A), and in response to an HLA-B27 restricted peptide in a HLA-B27 patient (FIG. 2B).

### EXAMPLE 3

### ELISpot Studies with AdBMI-1 Transduced Presenting Cells

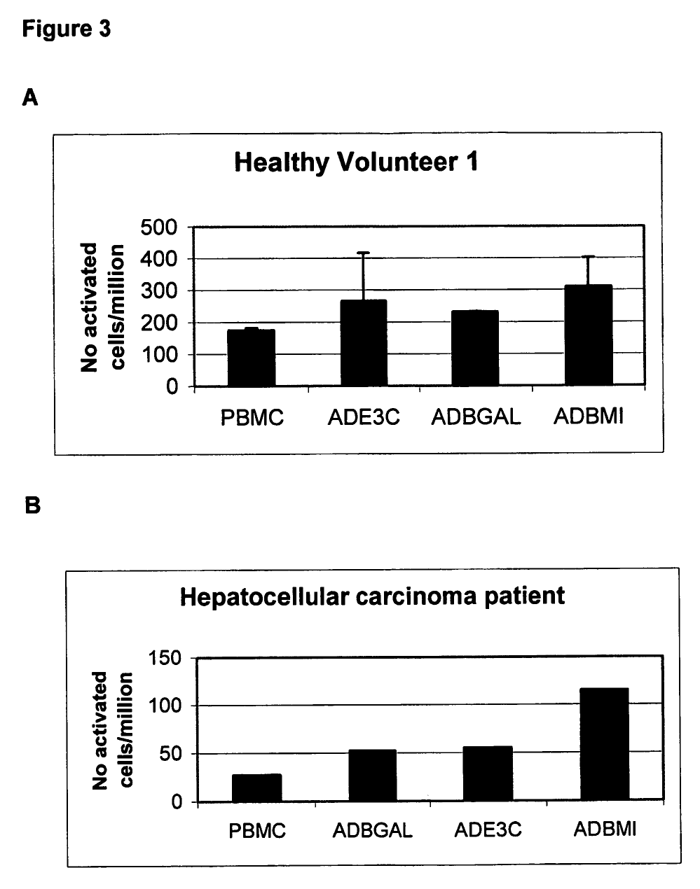

Elispots were performed using peripheral blood mononuclear cells (PBMCs) from an HCC patient and a normal donor. These assays were performed in duplicate where possible using 2-4×10⁵ cells per well. A replication-defective adenovirus containing the BMI-1 gene was produced in 293 cells and titred. PBMC were left to recover overnight if frozen samples were used, and then used in the ELISpot. Adenovirus was added at a MOI of 100. The plates were incubated overnight and then washed, antibodies added and stained according to usual protocols. The plates were counted using an automated system (AID, Strassburg, Germany). See FIG. 3.

### EXAMPLE 4

### Generation of Anti-BMI CTL In Vitro

Experiments are underway to generate CD8+ BMI-1 specific T cells by reactivation in vitro. A variety of restimulation protocols may be being employed, including the use of autologous dendritic cells or Langerhans cells, either pulsed with peptide epitopes or infected with the recombinant adenovirus expressing the whole protein. A novel method of stimulation labels autologous B cells with a streptavidin-conjugated anti-CD20 antibody followed by a biotin-labelled monomeric. HLA-A2/peptide complex.

Once successfully isolated, BMI-1 specific cytotoxic T lymphocytes are used to confirm that target cells endogenously processing the antigen are able to be efficiently recognised.

### EXAMPLE 5

### Prediction of HLA-A2 Epitopes in EZH2 and ELISpot Studies

Epitopes were predicted for EZH2 using the same protocol as Example 2.

| | | |
|---|---|---|
| A0201 peptides | YMCSFLFNL | (SEQ ID NO: 12) |
| and | | |
| | SQADALKYV. | (SEQ ID NO: 7) |
| B2702/05 peptides | KRFRRADEV | (SEQ ID NO: 13) |
| and | | |
| | YRYSQADAL. | (SEQ ID NO: 14) |
| B4402/03 peptides | EELFVDYRY | (SEQ ID NO: 15) |
| and | | |
| | KESRPPRKF. | (SEQ ID NO: 16) |

Elispots were carried out as described in Example 2.

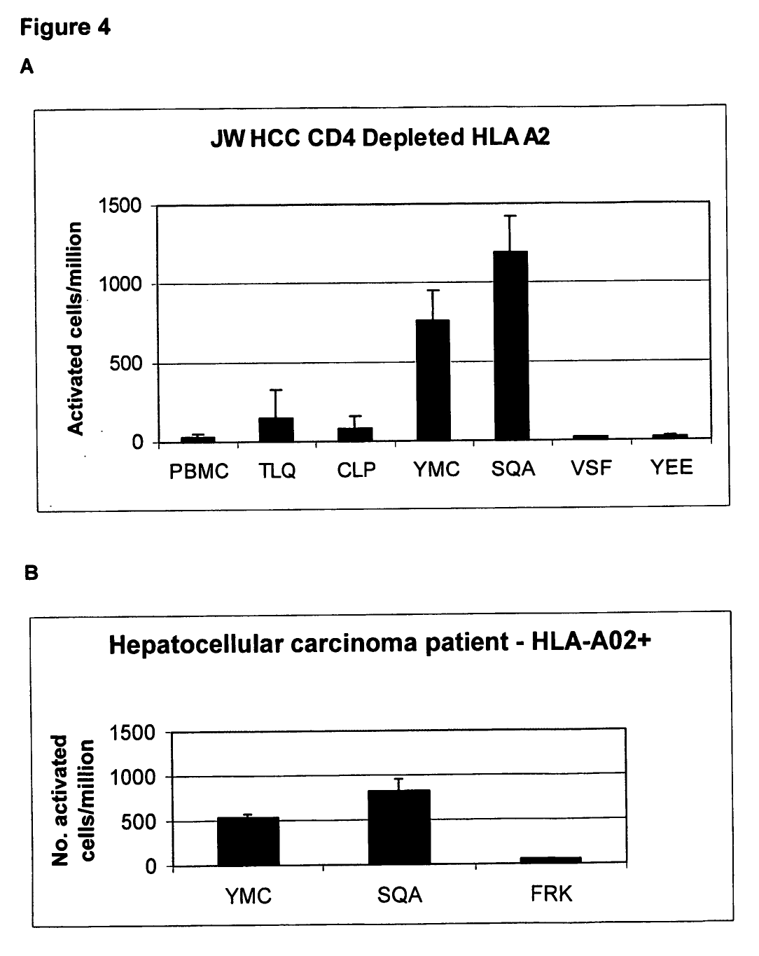

See FIG. 4.

### EXAMPLE 6

### Generation of Anti-BMI-1 Immune Responses in Murine Models

Mice are vaccinated against BMI-1. Methodologies for vaccination against an antigen are well known in the art and include, but are not limited to, the use of protein vaccines, peptide vaccines and genetic vaccines. Methodologies for genetic vaccination are well known to individuals skilled in the field and can include, but are not limited to, i) vaccination using BMI-1 encoding plasmid DNA (by needle injection using intradermal, subcutaneous or intramuscular routes), ii) gene gun delivery of BMI-1 encoding plasmid DNA (by the subcutaneous route), iii) by recombinant, BMI-1 encoding, viral vectors, an example of such a vector would be a first generation adenovirus encoding BMI-1, or iv) by a combination of the above approaches, for example the commonly used prime boost approach where the first administration, priming administration, is with naked DNA, and the subsequent boost is with a recombinant viral vector. Common immunisation protocols involve at least two administrations at least 6 days apart. Naked DNA administrations by a needle normally involve injection of 1-100 μg DNA by the intramuscular route. Gene Gun administrations use 01-10 μg of DNA delivered by the intradermal/subcutaneous route. When using a replication defective (E1/E3 deleted) adenovirus such as that described in Xiang et al. Virology (1996) 219:220-227, one can administer 1×10⁷ to 1×10¹⁰ pfu by the intramuscular, oral, intranasal or subcutaneous route. Following the last administration mice are usually left at least 5 days prior to harvesting serum to measure humoral responses or the spleen or lymph nodes to measure cellular responses. Humoral responses can be detected using a number of standard procedures well known to those skilled in the art. These methods include immunoblotting, Western blotting, a BMI-1 specific ELISA and radioimmunoprecipatation. These procedures are well-known to those skilled in the art. The following Western blotting protocol can be used to detect anti-BMI-1 antibodies in murine sera. Mice immunised with BMI-1 were bled at various time points following the vaccination. For each sample, protein extracts from a cell line expressing high amounts of BMI-1 protein (MCF-7) are run on NuPAGE Bis-Tris pre-cast gel (Invitrogen), and transferred to a PVDF membrane using the Xcell II Blot module (Invitrogen) according to manufacturer instructions (30V constant for 1 h in NuPAGE transfer Buffer+10% methanol). After 1 h blocking with casein at RT, membranes are blocked to avoid non-specific binding of biotin/avidin. This blocking step is performed using the Vector Blocking kit by 10 min incubation at RT in Avidin D solution (2 drops of Avidin D from the kit in 10 ml TBS) followed by a brief wash and 10 minutes incubation at RT in biotin solution (2 drops of Biotin from the kit in 10 ml TBS). After blocking, membranes are stained using VECTASTAIN ABC-AmP kit (Vector) according to the manufacturer's instructions. Briefly, membranes are incubated for 30 min with various dilutions of mouse serum in PBS and washed. For all washings, 3 incubations of 4 min in casein solution (Vector) are performed, and all incubations are done at RT. Membranes are then incubated for 30 min with 10 ml of biotinylated anti-mouse IgG (Vector) diluted at 1.5 μg/ml in casein solution. After washing, membranes are incubated 10 min with VECTASTAIN ABC-AmP Reagent diluted 1:500 in casein solution, washed, equilibrated by 5 min incubation in 0.1M Tris buffer pH 9.5, and stained with the Chromogenic Substrate Development kit (Vector, cat No. AK-6401). For this staining, membranes are incubated in the staining solution (4 drops of each reagent from the kit in 10 ml of 0.1M Tris buffer pH 9.5) for 5 to 30 min and washed with water. To detect the anti-BMI-1 antibodies by radio-immunoprecipitation following can be used. Labeled recombinant BMI-1 can be produced by in vitro transcription and translation (Promega, UK) with ³⁵S-methionine (Amersham, UK) from a plasmid encoding BMI-1 under the SP6 or T7 promoter. In vitro translated ³⁵S-BMI-1 (20,000 cpm) is incubated overnight at 4° C. with 2 μl of diluted hyperimmune murine serum (1:25 to 1:500). Autoantibody-bound antigen is precipitated with 25 μl of 25% protein A-Sepharose with 25% protein G-Sepharose (Amersham-Pharmacia, UK) in Multiscreen-DP opaque 96-well filtration plates (Millipore, UK) and is washed 8 times with washing buffer (20 mM Tris-HCl, pH 7.4, containing 150 mM NaCl, 0.1% BSA, and 0.15% Tween-20) using a Millipore vacuum-operated 96-well plate washer (Millipore, UK). After washing, scintillation fluid is added directly to the 96-well plate and radioactivity counted on a TopCount 96-well plate beta counter (Packard).

Cellular immune responses can be demonstrated by demonstrating T cell-derived antigen specific cytolytic activity or IFNγ secretion in response to BMI-1 presenting target cells. Spleens are removed from mice 10 days after the last BMI-1 immunisation. The spleen cells are immediately cocultured with syngenic Bone Marrow-derived Dendritic Cells (BMDC) transfected with a BMI-1 encoding construct using a non-viral transfection system, or electroporation. BMDC transfected with an irrelevant cDNA are used as negative controls, as well as spleen cells from mice immunised with irrelevant cDNA and adenovirus. Coculture is performed in ELispot plates (Millipore) coated with IFN-γ mAb with 5×10⁵ splenocytes/well and up to 1×10⁵ BMDC/well. After 24 h co-culture in 200 μl of RPMI 10% FCS, Elispot plates are washed and stained according to manufacturer instructions and spots are counted using digital image analyser software. BMDC are generated by culturing mouse BM cells (flushed from femurs and tibia), depleted of T cells and erythrocytes (cocktail of anti-CD4, -CD8, -CD45R followed by lysis with guinea pig complement and red cell lysis buffer.), in 500 U/ml murine GMCSF and 1000 U/ml murine IL-4.

### EXAMPLE 7

### Anti-tumour Responses in Murine Model(s)

Mice are vaccinated against BMI-1 using protocols and procedures as described in Example 6. The vaccinated mice are then challenged with a BMI-1 expressing syngeneic tumour cell line. The anti-tumour activity of the anti-BMI-1 immunisation is demonstrated by observed protection from challenge with the BMI-1 expressing tumour. An appropriate tumour model for this study is the murine mammary cell line 4T1 in Balb/c mice.

### EXAMPLE 8

### Immunostaining of Primary Tumour Samples

Method

Embedded tissue sections were stained as follows: the sections were soaked in xylene for 5 mins, alcohol for 5 mins, returned to water, and then treated with 0.3% H₂O₂ in H₂O for 15 mins followed by a wash in water. The sections were placed in EDTA buffer pH8 plus Tween20@ 65° C. on a hotplate stirrer (500 rpm) overnight (ALTER technique by G M Reynolds: Reynolds, G. M., Billingham, L. J., Gray, L. J., Flavell, J. R., Cocker, J., Scott, K., Young, L. S. and Murray, P. G. (2002) IL-6 expression in Hodgkin/Reed-Sternberg cells is associated with the presence of ‘B’ symptoms and failure to achieve complete remission in patients with advanced Hodgkin's disease. Br. J. Haematol. 118; 195-201). Slides were rinsed in water and were mounted onto a Sequenza™ (ThermoShandon, Runcorn, UK) and washed in TBS (Tris-buffered saline) pH 7.6. They were then incubated in primary anti-BMI-1 Ab (either a 1:10 dilution in TBS for 4 hrs for mC69 Ab (gift from Arie Otte, University of Amsterdam), or a 1:100 dilution in TBS for 1 hr for clone 229F6 mAb (Upstate Cell signalling Solutions, Milton Keynes, UK). Slides were then washed in TBS plus Tween20 and incubated in Dako EnVision™ secondary Ab (Dako UK Ltd, Ely, UK) for 30 mins. Slides were washed in TBS plus Tween20 and visualised by incubating in Vector NovaRED™ (Vector Laboratories, Peterborough, UK) for 10 mins. The slides were then counterstained in Mayers Haematoxylin for 10 secs, dehydrated, cleared and mounted.

Results

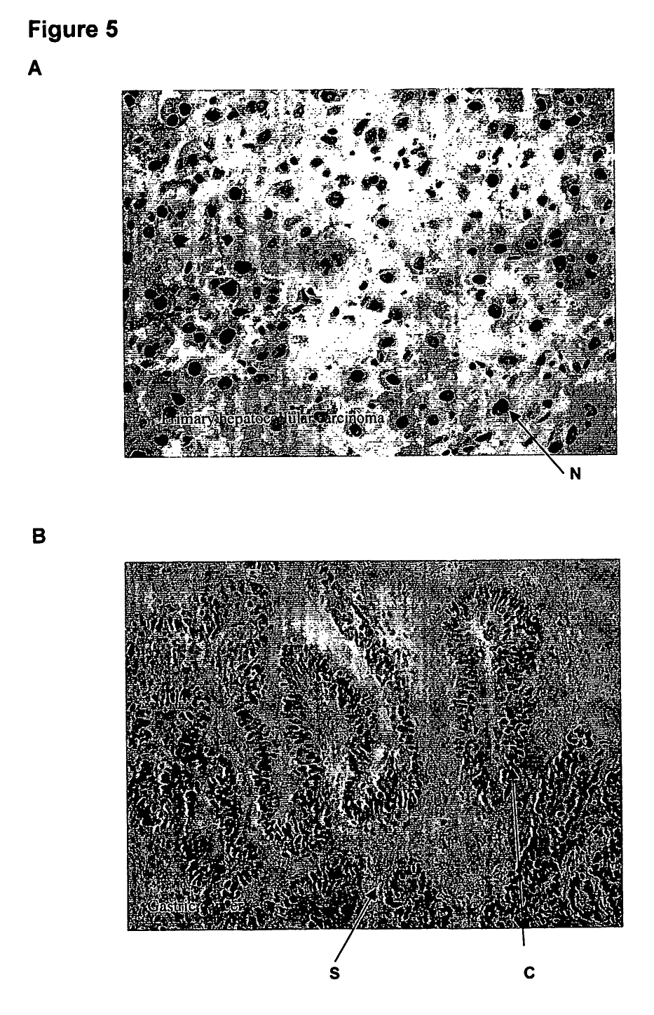

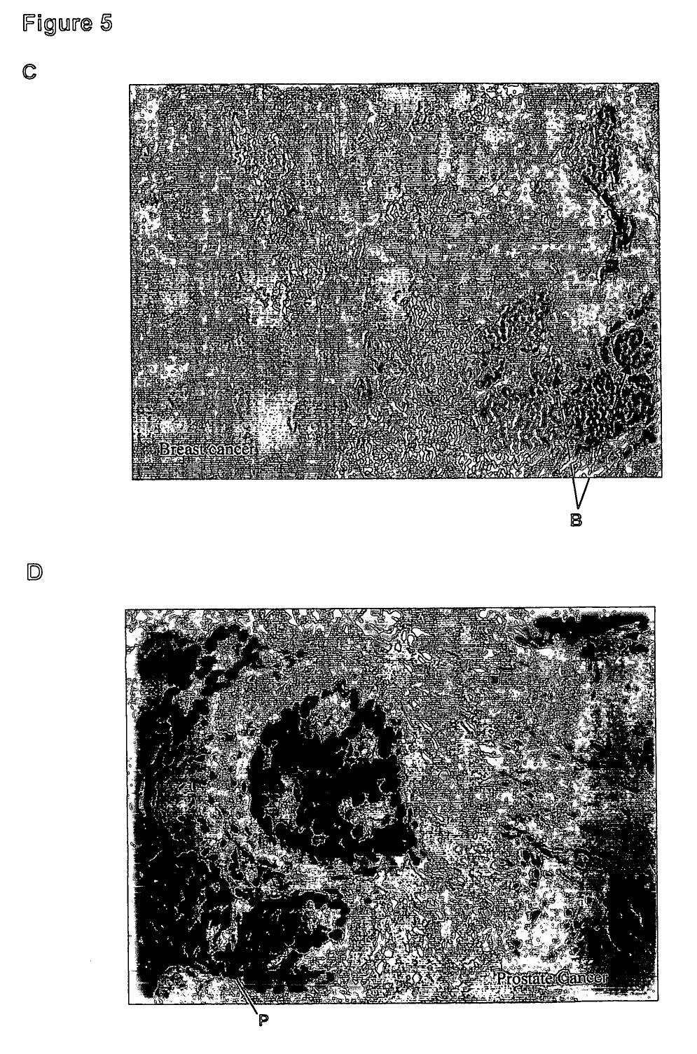

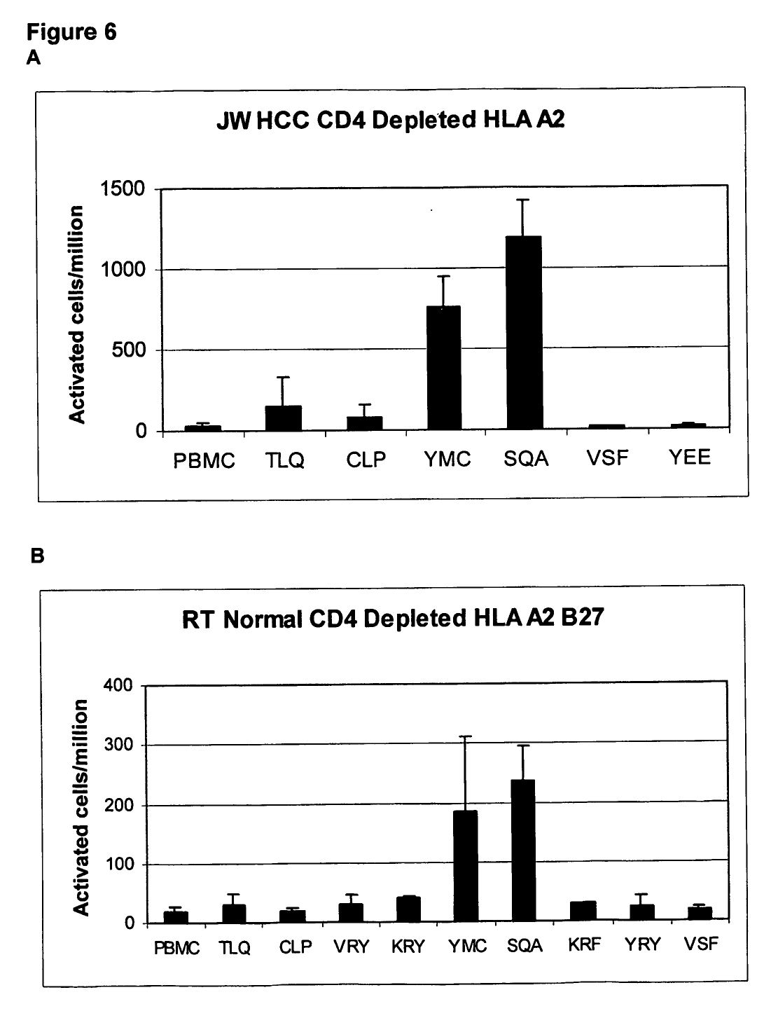

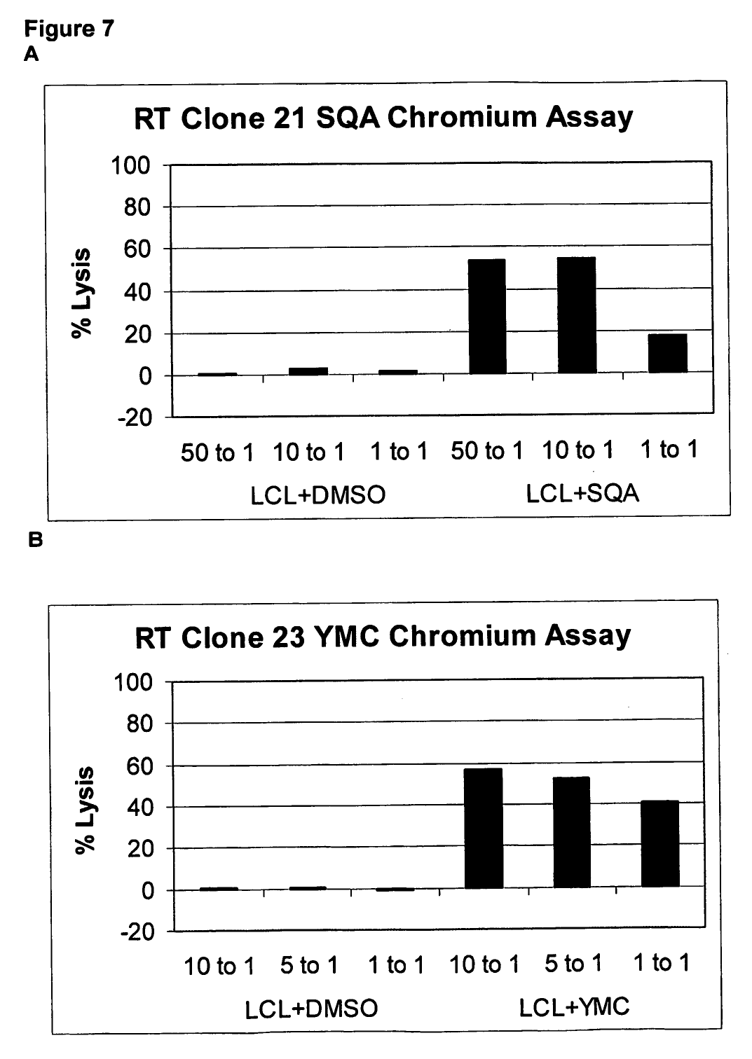

FIG. 5 shows the over-expression of BMI-1 in a number of tumour specimens. In all sections the nuclei of tumour cells can be seen to clearly stain positive for the presence of BMI-1. Positive staining tumour cells are indicated by an arrow(s) on each of the slides.

1-28. (canceled)

29. A host cell for the treatment of cancer that is transfected or tranduced with one or more of a polycomb protein, or an immunogenic peptide or epitope derived therefrom, or an isolated polynucleotide encoding said protein, peptide or epitope.

30. The host cell of claim 29, wherein one or more of said protein, peptide, or epitope comprises BMI-1, or an immunogenic peptide or epitope derived therefrom, or an isolated polynucleotide encoding said protein, peptide or epitope.

31. The host cell of claim 29, wherein one or more of said protein, peptide, or epitope comprises Enx/EZH2, or an immunogenic peptide or epitope derived therefrom, or an isolated polynucleotide encoding said protein, peptide or epitope.

32. A host cell for the treatment of cancer that is transfected or tranduced with at least one of a polycomb protein, or an immunogenic peptide or epitope derived therefrom, or an isolated polynucleotide encoding said protein, peptide or epitope, wherein the protein is selected from the group consisting of Enx/EZH2, EED, BMI-1, RING-1, HPH1, HPH2, HPC3 and CtBP.

33. The host cell of claim 29, 30, 31, or 32, wherein the cancer to be treated is selected from the group consisting of liver, lung, breast, stomach, colorectal, cervix, prostate, bladder, pancreas, brain or ovarian cancer, melanoma, lymphoma, or leukemia.

34. A vector for the treatment of cancer comprising at least one isolated polynucleotide encoding for a polycomb protein, or an immunogenic peptide or epitope derived therefrom.

35. The vector of claim 34, wherein said protein, peptide, or epitope comprises BMI-1, or an immunogenic peptide or epitope derived therefrom.

36. The vector of claim 34, wherein said protein, peptide, or epitope comprises Enx/EZH2, or an immunogenic peptide or epitope derived therefrom.

37. A vector for the treatment of cancer comprising at least one isolated polynucleotide encoding for a polycomb protein, or an immunogenic peptide or epitope derived therefrom, wherein the protein is selected from the group consisting of Enx/EZH2, EED, BMI-1, RING-1, HPH1, HPH2, HPC3 and CtBP.

38. The vector of claim 34, 35, 36, or 37, wherein the cancer to be treated is selected from the group consisting of liver, lung, breast, stomach, colorectal, cervix, prostate, bladder, pancreas, brain or ovarian cancer, melanoma, lymphoma, or leukemia.

39. A vaccine composition comprising:
one or more of a polycomb protein, or an immunogenic peptide or epitope derived therefrom, or an isolated polynucleotide encoding said protein, peptide or epitope, and
a pharmaceutically acceptable excipient, carrier, buffer or adjuvant.

40. The vaccine composition of claim 39,wherein one or more of said protein, peptide, or epitope comprises BMI-1, or an immunogenic peptide or epitope derived therefrom, or an isolated polynucleotide encoding said protein, peptide or epitope.

41. The vaccine composition of claim 39, wherein one or more of said protein, peptide, or epitope comprises Enx/EZH2, or an immunogenic peptide or epitope derived therefrom, or an isolated polynucleotide encoding said protein, peptide or epitope.

42. A vaccine composition comprising:
at least one polycomb protein, or an immunogenic peptide or epitope derived therefrom, or an isolated polynucleotide encoding said protein, peptide or epitope, and
a pharmaceutically acceptable excipient, carrier, buffer or adjuvant,
wherein the protein is selected from the group consisting of Enx/EZH2, EED, BMI-1, RING-1, HPH1, HPH2, HPC3 and CtBP.

43. A vaccine composition comprising:
a host cell transfected or tranduced with one or more of a polycomb protein, or an immunogenic peptide or epitope derived therefrom, or an isolated polynucleotide encoding said protein, peptide or epitope, and
a pharmaceutically acceptable excipient, carrier, buffer or adjuvant.

44. The vaccine composition of claim 43, wherein said protein, peptide, or epitope comprises BMI-1, or an immunogenic peptide or epitope derived therefrom.

45. The vaccine composition of claim 43, wherein said protein, peptide, or epitope comprises Enx/EZH2, or an immunogenic peptide or epitope derived therefrom.

46. A vaccine composition comprising:
a host cell transfected or tranduced with one or more of a polycomb protein, or an immunogenic peptide or epitope derived therefrom, or an isolated polynucleotide encoding said protein, peptide or epitope, and
a pharmaceutically acceptable excipient, carrier, buffer or adjuvant,
wherein the protein is selected from the group consisting of Enx/EZH2, EED, BMI-1, RING-1, HPH1, HPH2, HPC3 and CtBP.

47. A vaccine composition comprising:
a vector for the treatment of cancer comprising at least one isolated polynucleotide encoding for a polycomb protein, or an immunogenic peptide or epitope derived therefrom, and
a pharmaceutically acceptable excipient, carrier, buffer or adjuvant.

48. The vaccine composition of claim 47, wherein said protein, peptide, or epitope comprises BMI-1, or an immunogenic peptide or epitope derived therefrom.

49. The vaccine composition of claim 47, wherein said protein, peptide, or epitope comprises Enx/EZH2, or an immunogenic peptide or epitope derived therefrom.

50. A vaccine composition comprising:
a vector for the treatment of cancer comprising at least one isolated polynucleotide encoding for a polycomb protein, or an immunogenic peptide or epitope derived therefrom, and
a pharmaceutically acceptable excipient, carrier, buffer or adjuvant,
wherein the protein is selected from the group consisting of Enx/EZH2, EED, BMI-1, RING-1, HPH1, HPH2, HPC3 and CtBP.

51. A method of treating a cancer by immunotherapy, comprising administering to a patient a vaccine composition comprising a polycomb protein, or an immunogenic peptide or epitope derived therefrom, or an isolated polynucleotide encoding said protein, peptide or epitope.

52. The method according to claim 50, wherein the protein is selected from the group consisting of Enx/EZH2, EED, BMI-1, RING-1, HPH1, HPH2, HPC3 and CtBP.

53. The method according to claim 51, wherein the protein is BMI-1.

54. The method of claim 50, 51, or 52, wherein the cancer to be treated is derived from a tissue or organ selected from the group consisting of, liver, lung, breast, stomach, cervix, prostate, bladder, pancreas, brain, colorectal or ovarian cancer, melanoma, lymphoma or leukemia.

55. A method of treating a cancer by immunotherapy, comprising administering to a patient a vaccine composition comprising a polynucleotide encoding a polycomb protein, or an immunogenic peptide or epitope derived therefrom.

56. The method of claim 54, wherein the polynucleotide is included in a vector.

57. The method of claim 55, wherein the vector is an integrating vector.

58. The method of either of claim 56 wherein the vector is a viral vector.

59. The method of claim 55, wherein the vector is a non-integrating vector.

60. The method of claim 58, wherein the vector is a viral vector.

61. The method according to claim 54, 55, 56, 57, 58, or 59, wherein the protein is selected from the group consisting of Enx/EZH2, EED, BMI-1, RING-1, HPH1, HPH2, HPC3 and CtBP.

62. The method according to claim 60, wherein the protein is BMI-1.

63. The method according to claim 60, wherein the protein is Enx/EZH2.

64. A method of treating a cancer by immunotherapy, comprising administering to a patient a vaccine composition comprising a host cell containing a polycomb protein, or an immunogenic peptide or epitope derived therefrom, or an isolated polynucleotide or vector encoding said protein, peptide or epitope.

65. The method of claim 63, wherein said host cell is a dendritic cell.

66. The method of claim 63 or 64, wherein the cancer to be treated is derived from a tissue or organ selected from the group consisting of liver, lung, breast, stomach, cervix, prostate, bladder, pancreas, brain, colorectal or ovary, or is a melanoma, lymphoma or leukemia.
